(19) United States
(12) Patent Application Publication
(10) Pub. No.: US 2023/0346210 A1
(43) Pub. Date: Nov. 2, 2023

(54) Combined Mirror Holder and Suction Device

(71) Applicant: Ajay Potluri, Surrey (CA)

(72) Inventor: Ajay Potluri, Surrey (CA)

(21) Appl. No.: 18/245,429

(22) PCT Filed: Sep. 22, 2021
(86) PCT No.: PCT/CA2021/051315

§ 371 Date: Mar. 15, 2023

Related U.S. Application Data
(60) Provisional application No. 63/081,605, filed on Sep. 22, 2020.

(51) Int. Cl. A61B 1/247; A61B 1/12; A61B 1/00; A61C 17/06

(52) U.S. Cl. CPC A61B 1/247; A61B 1/126; A61B 1/00094; A61C 17/088

(57) ABSTRACT

A multi-purpose dental tool for simultaneous fluid evacuation and mirror support. An elongated tool body with a hollow longitudinal passage has a hose connector at a proximal end, and suctions ports near an opposing distal end. The distal end features a concavely arcuate seating edge in a plane of oblique relation to the body's longitudinal axis for seated placement of a mirrored tip of a replaceable minor head. A receiving cavity situated on an underside of the tool body near the distal end receives a stem of the mirror head. An enlarged gripping portion resides near a control valve of the tool, and the hose connector swivels to prevent hose kinking. Two primary suction ports are provided in a concave topside of the tool body at a flared region approaching the seating edge. Two smaller slot-shaped secondary ports penetrate the underside of the tool body near the seating edge.

### FIELD OF THE INVENTION

The present invention relates generally to dentistry tools, and more particularly to dual-purpose devices designed to both evacuate liquids from a dental patient's oral cavity and simultaneously support a manipulatable dental mirror therein in a movable manner.

### BACKGROUND

In the field of dentistry, multi-purpose dental tools for simultaneous fluid evacuation and mirror support in a patient's oral cavity have been previously proposed, both in the prior patent literature and in the commercial marketplace, of which commercially available example is the Purevac HVE System from Densply Sirona. Despite this, there remains room for improvements and alternatives to the previously proposed tools.

Disclosed herein is Applicant's new proposal for a combined dental mirror and suction tool, which embodies novel features and combinations that, to best of Applicant's knowledge, are neither heretofore known, nor suggested by the prior art.

### SUMMARY OF THE INVENTION

According to a first aspect of the invention, there is provided a combined mirror holder and suction device for dental applications, said device comprising:
an elongated tool body having a proximal end, a distal end residing opposite thereto in a longitudinal direction of said tool body denoted by a longitudinal axis thereof, said tool body having opposing top and bottom sides residing oppositely of one another in a thickness direction orthogonally transverse to said longitudinal axis, and opposing lateral sides residing opposite one another in a width direction orthogonally transverse to both said longitudinal axis and said first direction;
a hollow internal passage running longitudinally and internally of said tool body on the longitudinal axis thereof;
one or more suction ports fluidly communicating said hollow internal passage with an exterior of the tool body at or adjacent the distal end thereof, of which at least one of said one or more suction ports resides at the top side of the tool body; and
a hose connector residing at or adjacent the proximal end of the elongated tool body and fluidly communicating with said hollow internal passage to enable connection of a suction hose from a vacuum pump to apply suction at said suction port;
wherein for the purpose of removably supporting a replaceable dental mirror head that is composed of stem and a mirrored tip attached to one end of said stem, said distal end of the tool body comprises a concavely arcuate seating edge residing in a reference plane of obliquely oriented relation to the longitudinal axis of the tool body to accept seated placement of an outer circumference of the mirrored tip of the mirror head against said curved seating edge in a position facing a reflective surface of said mirrored tip outwardly away from the top side of the tool body.

According to a second aspect of the invention, there is provided a combined mirror holder and suction device for dental applications, said device comprising:
an elongated tool body having a proximal end, a distal end residing opposite thereto in a longitudinal direction of said tool body denoted by a longitudinal axis thereof, said tool body having opposing top and bottom sides residing oppositely of one another in a thickness direction orthogonally transverse to said longitudinal axis, and opposing lateral sides residing opposite one another in a width direction orthogonally transverse to both said longitudinal axis and said first direction;
a hollow internal passage running longitudinally and internally of said tool body on the longitudinal axis thereof;
one or more suction ports fluidly communicating said hollow internal passage with an exterior of the tool body at or adjacent the distal end thereof, of which at least one of said one or more suction ports resides at the top side of the tool body; and
a hose connector residing at or adjacent the proximal end of the elongated tool body and fluidly communicating with said hollow internal passage to enable connection of a suction hose from a vacuum pump to apply suction at said suction port; and
for the purpose of removably supporting a replaceable dental mirror head that is composed of stem and a mirrored tip attached to one end of said stem, a longitudinally oriented receiving cavity situated on the bottom side of the tool body at a proximally spaced distance from the distal end thereof;
wherein said receiving cavity has an open end facing longitudinally toward the distal end of the tool body to accommodate the stem of the mirror head, and is expandable and collapsible in diameter between an expanded state accommodating insertion of the stem of the mirror head, and a normal state of lesser diameter than the stem of the mirror head to exert frictional holding force thereon when received in the cavity.

According to a third aspect of the invention, there is provided a combined mirror holder and suction device for dental applications, said device comprising:
an elongated tool body having a proximal end, a distal end residing opposite thereto in a longitudinal direction of said tool body;
a hollow internal passage running longitudinally and internally of said tool body;
at least one suction port fluidly communicating said hollow internal passage with an exterior of the tool body at or adjacent the distal end thereof;
a hose connector residing at or adjacent the proximal end of the elongated tool body and fluidly communicating with said hollow internal passage to enable connection of a suction hose from a vacuum pump to apply suction at said suction port; and
a control valve installed on said tool body at an intermediate location along the hollow passage, said control valve comprising an external manipulator situated outside the tool body and a valve body that resides within the hollow passage and is movable via the external manipulator to vary airflow through the hollow passage;
wherein:
   the tool body, at or adjacent the distal end thereof, comprises a mirror holding portion that holds or is configured to hold a mirror in a working position facing outwardly from the top side of the tool body at or adjacent the distal end thereof; and
   the tool body, at a gripping portion thereof that proximally neighbours the control valve and spans longitudinally therefrom toward the proximal end of the tool body, has an enlarged circumference relative to a joining portion of the tool body that distally neighbours the control valve and spans longitudinally therefrom toward the distal end of the tool body.

According to a fourth aspect of the invention, there is provided a dental suction device comprising:
an elongated tool body having a proximal end, a distal end residing opposite thereto in a longitudinal direction of said tool body;
a hollow internal passage running longitudinally and internally of said tool body;
at least one suction port fluidly communicating said hollow internal passage with an exterior of the tool body at or adjacent the distal end thereof; and
a hose connector residing at or adjacent the proximal end of the elongated tool body and fluidly communicating with said hollow internal passage to enable connection of a suction hose from a vacuum pump to apply suction at said suction port; and
wherein said hose connector is coupled to the tool body in swiveling fashion to enable tilting of said hose connector in multiple directions relative to the longitudinal axis of the tool body.

According to a fifth aspect of the invention, there is provided a combined mirror holder and suction device for dental applications, said device comprising:
an elongated tool body having a proximal end, a distal end residing opposite thereto in a longitudinal direction of said tool body denoted by a longitudinal axis thereof, said tool body having opposing top and bottom sides residing oppositely of one another in a thickness direction that is orthogonally transverse to said longitudinal axis, and opposing lateral sides residing opposite one another in a width direction that is orthogonally transverse to both said longitudinal axis and said first direction;
a hollow internal passage running longitudinally and internally of said tool body on the longitudinal axis thereof;
a plurality of suction ports fluidly communicating said hollow internal passage with an exterior of the tool body near the distal end thereof; and
a hose connector residing at or adjacent the proximal end of the elongated tool body and fluidly communicating with said hollow internal passage to enable connection of a suction hose from a vacuum pump to apply suction at said plurality of suction ports;
wherein:
   the tool body, at or adjacent the distal end thereof, comprises a mirror holding portion that holds or is configured to hold a mirror in a working position facing outwardly from the top side of the tool body at or adjacent the distal end thereof;
   said plurality of suction ports comprises a pair of primary suction ports penetrating a top wall of the tool body on the top side thereof at a location situated adjacent to the mirror holding portion at a proximal side thereof nearest to the proximal end of the tool body.

According to a sixth aspect of the invention, there is provided a combined mirror holder and suction device for dental applications, said device comprising:
an elongated tool body having a proximal end, a distal end residing opposite thereto in a longitudinal direction of said tool body denoted by a longitudinal axis thereof, said tool body having opposing top and bottom sides residing oppositely of one another in a thickness direction that is orthogonally transverse to said longitudinal axis, and opposing lateral sides residing opposite one another in a width direction that is orthogonally transverse to both said longitudinal axis and said first direction;
a hollow internal passage running longitudinally and internally of said tool body on the longitudinal axis thereof;
a plurality of suction ports fluidly communicating said hollow internal passage with an exterior of the tool body near the distal end thereof; and
a hose connector residing at or adjacent the proximal end of the elongated tool body and fluidly communicating with said hollow internal passage to enable connection of a suction hose from a vacuum pump to apply suction at said plurality of suction ports;
wherein:
   the tool body, at or adjacent the distal end thereof, comprises a mirror holding portion that holds or is configured to hold a mirror in a working position facing outwardly from the top side of the tool body at or adjacent the distal end thereof;
   at a region of said tool body approaching and connected to said mirror-holding portion, the tool body is further characterized by at least one of the following features:
(a) a concave external curvature at the top side of the tool body;
(b) a convex external curvature at the bottom side of the tool body;
(c) a tapered shape by which a depth of the internal passage tapers toward the distal end of the tool body; and/or
(d) a flared shape by which a combined wall height of the bottom and lateral sides of the tool body measured in the thickness direction increases toward the distal end of the tool body.

### BRIEF DESCRIPTION OF THE DRAWINGS

One embodiment of the invention will now be described in conjunction with the accompanying drawings in which:

FIG. 1 is a top perspective view of a combined mirror holder and suction device for dental applications, as viewed from a first side thereof.

FIG. 2 is another top perspective view of the device of FIG. 1, but from a second side thereof.

FIG. 3 is an exploded top perspective view of the device of FIG. 1 from the second side thereof.

FIG. 4 is a partial side view of the device of FIG. 1, but with a replaceable mirror head of the device removed for illustrative purpose.

FIG. 5 is a partial top plan view of the device of FIG. 4 showing a mirror holding portion at a distal end thereof.

FIG. 6 is a partial bottom plan view of the mirror holding portion of FIG. 5.

FIG. 7 is an end view of the device of FIG. 4 from the distal end thereof at which the removable mirror head is held when installed thereon.

FIG. 8 is a transverse cross section of the device as viewed along line A-A of FIG. 5.

FIG. 9 is another transverse cross section of the device, but as viewed along line B-B of FIG. 5.

FIG. 10 is a longitudinal cross-section of transverse cross section of the device, as viewed along line C-C of FIG. 6.

FIG. 11 is a bottom perspective view of the combined mirror holder and suction device of FIG. 1.

FIG. 12 is a longitudinal cross-section of the mirror holding portion of FIG. 5 as viewed along line D-D thereof.

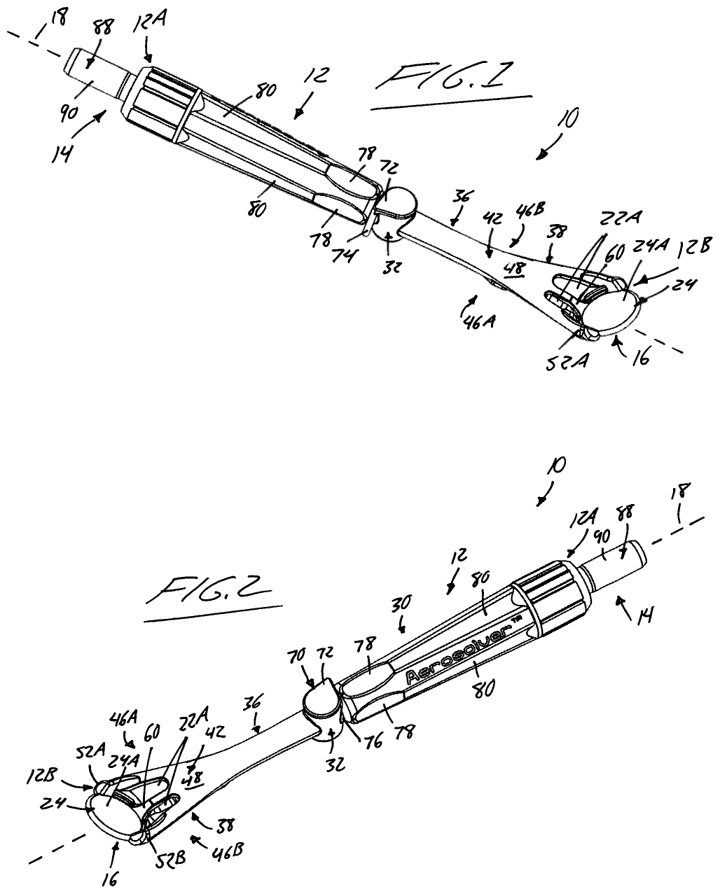

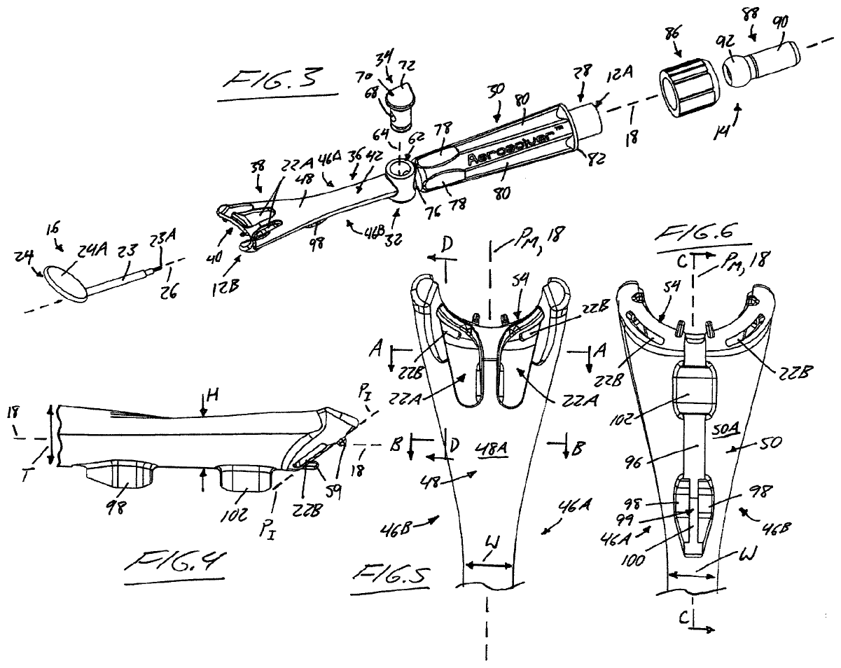

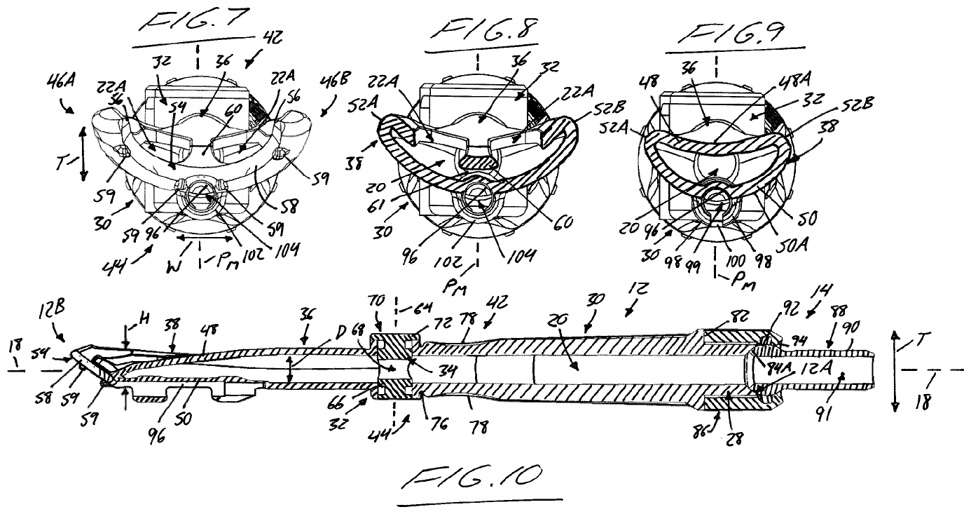

### DETAILED DESCRIPTION

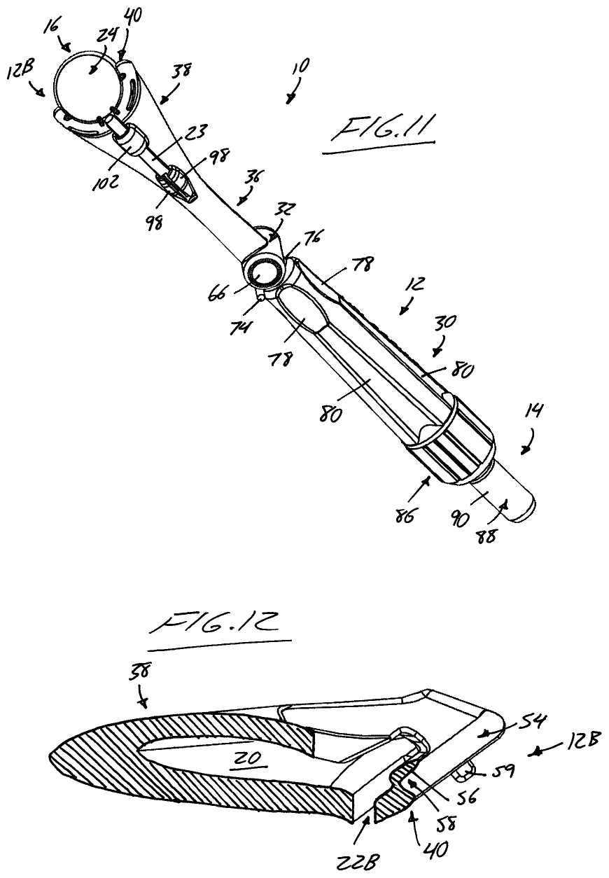

The combined mirror holder and suction device 10 shown in the appended drawings is composed primarily of an elongated tool body 12 having longitudinally opposed proximal and distal ends 12A, 12B, a hose connector 14 mounted to the tool body 12 at the proximal end 12A of the tool body 12, and a removable/replaceable mirror head 16 removably supported at the distal end 12B of the tool body. A central longitudinal axis 18 of the tool body 12 denotes the longitudinal direction in which the proximal and distal ends 12A, 12B of the tool body 12 are spaced apart from one another, and is used as a reference axis to describe the relative locations and orientations of other features of the device. As best seen in the longitudinal cross-section of FIG. 10, which is taken in a longitudinal midplane P_{M} of the tool body 12 that contains the central longitudinal axis 18 and bisects the tool body 12 into two symmetrical halves, the tool body 12 is of hollow tubular structure over most of its longitudinally measured length. A hollow internal passage 20 of the tool body 12 runs along the central longitudinal axis from the proximal end 12A of the tool body to a location near the opposing distal end 12B thereof.

Near the distal end 12B of the tool body, a set of suction ports 22A, 22B penetrate the tool body exterior and open into the hollow internal passage 20. The hose connector 14 enables connection of a suction hose (not shown) from a vacuum pump (not shown) to the proximal end 12A of the tool body in a manner fluidly communicating the suction hose with an open end of the internal passage 20 at the proximal end 12A of the tool body. Under operation of the vacuum pump, suction is thus created at the suction ports 22A, 22B for the purpose of collective fluids (saliva, water, blood) from the oral cavity of a dental patient when the device's distal end 12B is inserted into said oral cavity. As described in more detail below, the distal end 12B of the tool body is specially configured to removably support the replaceable mirror head 16, which features an elongated stem 23 that carries a circular mirrored tip 24 at one end of the stem. The mirrored tip 24 is of notably greater diameter than the relatively narrow stem 23, and resides at an inclined plane of oblique relationship to a central linear axis 26 of the stem. The mirrored tip has a reflective surface 24A on the side thereof facing away from the linear axis, and resides at an obtusely oblique angle to the stem. The stem 23 and its linear axis 26 lie parallel to the longitudinal axis 18 of the tool body when the mirror head 16 is installed on the tool body. As shown in FIG. 3, the mirror head 16 may be of a conventional, commercially available type where a terminal end 23A of the stem 23 opposite the mirrored tip 24 is externally threaded, where such threading is conventionally used to mount the replaceable mirror head to a handle of a single-purpose mirror tool of a conventional type lacking any suction capability. Alternatively, the mirror head 16 may be specially produced for the device of the present invention, in which case such threading of the stem's terminal end 23A is unnecessary, and may be omitted.

The tool body 12 has a number of distinctly shaped sections arranged in series with one another in the longitudinal direction of the tool body. Listed sequentially starting from the proximal end 12A, these sections include a coupling portion 28 configured for attachment of the suction hose (via the separate hose connector 14, in the illustrated example), a grip portion 30 configured for manual gripping by the user of the device, a valve portion 32 equipped with a user-operable control valve 34 for controlling airflow and suction through the internal passage 20 of the tool body 12, a joining portion 36 spanning longitudinally from the valve portion 32 toward the distal end 12B of the tool body 12, a flared and tapered portion 38 that features the suction ports 22A, 22B and is connected to the valve portion 32 by the joining portion 36, and finally a mirror holding portion 40 at the distal end 12B where the mirrored tip 24 of the mirror head is supported when the mirror head 16 is installed. All portions other than the final mirror holding portion 40 are hollow parts of the tool body 12 through which the internal passage 20 extends in order to fluidly communicate the suction ports 22A, 22B with the vacuum hose, except when the user-operable valve 34 is placed in a fully closed position preventing airflow through the tool body 12 from the suction ports 22A, 22B to the connected suction hose.

In a circumferential direction around the linear axis 18, the tool body 12 is considered to have four different sides: a top side 42 and a bottom side 44 that resides oppositely of one another in a thickness direction T that is measured orthogonally of the longitudinal axis 18 within the longitudinal midplane; and two lateral sides 46A, 46B that resides oppositely of one another in a width direction W that is measured orthogonally to both the longitudinal axis 18 and the thickness direction T, and normally to the longitudinal midplane P_{M}.

At the flared and tapered portion 38, the two lateral sides 46A, 46B of the tool body 12 diverge away from one another toward the distal end 12B of the tool body in symmetric fashion about the longitudinal midplane P_{M}, whereby the width W of the tool body 12 increases form the narrower joining portion 36 to the mirror holding portion 40. On the top side 42 of the tool body 12, the hollow internal passage 20 is bounded by a respective top wall 48 of the tool body. On the opposing bottom side 44 of the tool body 12, the hollow internal passage 20 is bounded by a respective bottom wall 50 of the tool body that lies opposite the top wall 48 in the thickness direction T. The suction ports 22A, 22B include two primary suction ports 22A that penetrate the top wall 48 and open into the hollow internal passage 20, and two secondary suction ports 22B that penetrate the bottom wall 50 and also open into the hollow internal passage 20. As best shown in FIG. 9, at the flared/tapered portion 38 of the tool body, the top wall's exterior surface 48A that faces outwardly from the tool body 12 is concavely arcuate, while the bottom wall's exterior surface 50A that faces outwardly from the tool body is convexly arcuate. The two lateral sides 46A, 46B of the tool body at the flared and tapered portion 38 thereof have smoothly rounded external corners 52A, 52B that join together the concave and convex exteriors of the top and bottom walls 48, 50 in seamlessly integral and continuously curved fashion. The top and bottom walls and rounded corners of the flared/tapered portion 38 are of generally uniform thickness throughout, whereby their interior surfaces are of opposite concavity/convexity to their exterior surfaces. Accordingly, the interior passage 20 is flared in width toward the distal end of the tool body at the flared/distal portion thereof, just like the tool body's exterior.

The distal end 12B of the tool body is at least partially embodied by a terminal edge 54 of the externally convex bottom wall 50. This terminal edge 54 of the bottom wall 50 resides in an inclined plane P_{I} of oblique orientation to the longitudinal axis 18 of the tool body 12 at an oblique orientation of matching obtuse angle to that which exists between the mirror tip 24 and stem 23 of the mirror head 16. Within this inclined plane P_{I}, the terminal edge 54 of the bottom wall 50 has a concavely arcuate shape that is bisected by the longitudinal midplane P_{M} of the tool body 12. In radial cross-sectional planes of the terminal edge 54 (i.e. planes lying radially of its arcuate curvature), the terminal edge 54 has a notched profile creating a slightly protruding retaining lip 56 at the top side of this edge. The lip 56, best seen in FIG. 12, overhangs an undercut seating notch 58 that continuously spans the full arcuate length of the terminal edge 54, thereby defining to an arcuate seating surface mfor conformingly abutted seating thereagainst of the circular outer perimeter of the mirror head's mirrored tip 24.

At spaced intervals along this seating surface 58 at the bottom side of the bottom wall's terminal edge 54 are a set of support nubs 59 projecting outwardly from said concavely arcuate seating surface 58 to brace against a backside of the mirror head's mirrored tip 24 (i.e. the side thereof opposite the reflective surface 24A) when the outer perimeter of the mirror head's mirrored tip 24 is seated against the seating surface 58. At the same time, the retaining lip 56 of the terminal edge 54 reaches slightly over the outer perimeter of the mirror head's mirrored tip 24 on the opposing reflective frontside thereof, whereby the mirrored tip 24 is securely captured between the retaining lip 56 and supporting nubs 59 to prevent deflection of the mirrored tip 24 out its seated position against the concavely arcuate seating surface 58. The concave terminal edge 54 and its seating surface 58, retaining lip 56 and support nubs 59 thus collectively define the mirror holding portion 40 of the tool body 12 by which the mirrored tip 24 of a removable/replaceable mirror head 16 is supported on the tool body in the illustrated embodiment.

The primary suction ports 22A penetrate the top wall 48 of the flared/tapered portion 38 of the tool body 12 at locations situated immediately or closely adjacent the concavely curved terminal edge 54 of the mirror holding portion of the tool body, thus immediately neighbouring the mirror holding portion 40 on the proximal side thereof. The two primary suction ports 22A are symmetrically disposed to one another across the longitudinal midplane P_{M}, and are separated from one another by an intact strip of the top wall 48 that spans longitudinally between the two ports 22A at the longitudinal midplane P_{M}, thereby defining a dividing rib 60 between the two ports. A distal end of this divider rib 60 joins up with the bottom wall 50 of the tool body at a center point of the concavely arcuate terminal edge 54 thereof. In the illustrated example, the two primary suction ports 22A are of elongated shape having greater length in the longitudinal direction of the tool body than in the width direction W thereof. The two secondary suction ports 22B penetrate the bottom wall 50 of the flared/tapered portion 38 of the tool body 12 at positions also residing closely or immediately adjacent the concavely curved terminal edge 54 of the bottom wall 50 at the proximal side of the mirror holding portion 40 of the tool body 12. The secondary suction ports 22B in the illustrated example are narrow slot like openings, whose elongated dimensions, instead of lying longitudinally of the tool body 12 like the primary suction ports 22A, instead are oriented in more widthwise transverse fashion to the tool body 12 and its longitudinal axis 18. Each secondary suction port 22B has an arcuate curved slot shape spanning in conforming fashion along a respective segment of the arcuately concave terminal edge 54 of the mirror holding portion 40 of the tool body 12. Like the primary suction ports 22A, the secondary suction ports 22B are shaped and positioned symmetrically of one another across the longitudinal midplane P_{M} of the tool body 12. Each secondary suction port 22B underlies a respective one of the primary suction ports 22A at or near a distal end thereof nearest to the arcuately concave terminal edge 54. Both the primary and secondary suction ports 22A, 22B therefore reside closely or immediately adjacent to the mirror holding portion 40 of the tool body at the proximal side thereof, and are open to one another within the internal passage 20 at a distal end thereof that closely neighbours the mirrored tip 24 of the removable/replaceable mirror head 16. The divider rib 60 is connected to the bottom wall 50 of the tool body 12 only at the divider rib's distal end, whereby the internal passage 20 is not bifurcated into two separated channels by the divider rib 60. The two primary suction ports 22A, and the two secondary suction ports 22B, are therefore fluidly communicated with one another via a gap space 61 between the bottom wall 50 and the divider rib 60 of the top wall 48, as seen in FIG. 8.

The larger primary suction ports 22A at the concave top side 42 of the tool body's flared/tapered portion 38 are typically used to suction fluids from the patient's oral cavity, but the smaller secondary suction ports 22B at the convex bottom side 44 can also be used for such purpose, for example at harder to reach areas of the oral cavity that are less accessible by the concave top side 42. Also, should the patient's tongue or lip be caught in a position fully obstructing the primary suction ports 22A, the overall suction force applied to the captured tissue is lessened by the admission of some airflow through the smaller secondary suction ports 22B. The use two neighbouring but separated primary suction ports 22A, rather than a larger singular primary suction port, is believed to provide or contribute reduced suction noise relative to alternative designs.

The flared shape of the flared/tapered portion 38 of the tool body 12 refers both to internal and external flaring thereof in the width direction W toward the distal end of the tool body, and an external flaring thereof in the thickness direction T toward the distal end of the tool body. The flaring of the internal and external width of the tool body is denoted by the divergent relationship between the rounded corners 52A, 52B at the two lateral sides 46A, 46B of this portion 38 as they extend toward the distal end 12B of the tool body, and the corresponding widening of the externally convex bottom wall 50 in this same direction. Both the overall external width of the tool body, and the interior width measured across the internal passage 20, thus increase in flared fashion toward the distal end 12B of the tool body. The flaring of the tool body thickness T refers to variation in a combined wall height H of the externally convex bottom wall 50 and the two rounded corners 52A, 52B, as measured in the thickness direction T. This combined wall height H increases to a maximum at the distal end 12B of the tool body 12, where this increased wall height is used to give the terminal edge 54 an approximate 180-degree perimeter span to encapsulate an approximately full semi-circular half of the mirrored tip 24 of the mirror head 16, thereby providing robust, stable support thereof.

The tapered characteristic of the flared/tapered portion 38 on the other hand is refers to varying measure in the thickness direction T between the top and bottom walls 48, 50, and the resulting variation in a depth D of the internal passage 20 measured between said top and bottom walls 48, 50. This internal depth D is tapered in longitudinally decreasing fashion toward the distal end 12B of the tool body, as best seen in the longitudinal cross section of FIG. 10, where the top wall 48 at the flared/tapered portion 38 lies obliquely to the longitudinal axis 18 of the tool body to converge toward the bottom wall 50, which remains substantially parallel to the longitudinal axis 18 throughout the length of the flared-tapered portion, before angling upward at the inclined plane P_{I} at the mirror holding portion 40. The tapered and flaring aspects of the tool body shape occur only the flared/tapered portion thereof at which the suction ports 22A, 22B reside. The adjacent joining portion 36 that extends longitudinally from the flared/tapered portion 38 to the valve portion 32 is neither flared nor tapered in either of the width and thickness directions, instead being uniform in terms of both its internal passage depth and width, and its external thickness and width.

Running axially along the bottom side of the flared/tapered portion 38 of the tool body over a full or substantial length thereof in parallel relation to the longitudinal axis is a longitudinal receiving channel 96 that is recessed into the bottom wall 50 at the longitudinal midplane P_{M} of the tool body 12. The receiving channel 96, in cross-sectional planes normal to the longitudinal axis 18, is concavely arcuate, with an arcuate span of 180-degrees or less, and has a radius of curvature equal to or slightly exceeding that of the stem 23 of the mirror head 16. At or near the junction of the flared/tapered portion 38 and the neighbouring joining portion 36, the bottom side 44 of the tool body 12 features a pair of resiliently flexible grippers 98 for frictionally gripped capture of the mirror head stem 23 in a receiving cavity 99 that is cooperatively defined by said receiving channel 96 and said grippers 98. The grippers 98 neighbour the channel 96 on opposing sides thereof at or near a proximal end thereof, and reside in symmetric relation to one another across the longitudinal midplane P_{M} that bisects the receiving the channel 96.

Interior surfaces of the grippers 98 are concavely arcuate surfaces forming continuous extensions of the receiving channel's arcuate wall shape, whereby the channel and the inner surfaces of the grippers collectively define a split wall structure spanning more than 180-degrees, but less than 360-degrees, around an axis of channel 96 to substantially enclose a receiving cavity 99 of round cross-section. In a default state of this receiving cavity 99, an inner diameter thereof is slightly less than an outer diameter of the mirror head stem 23, but through flexing of the two grippers 98 away from another to enlarge the width of a strip-shaped gap 100 therebetween, the cavity is enlargeable into an expanded state of greater inner diameter equal to or exceeding the outer diameter of the mirror head stem 23. To install the mirror head, the free end of the stem 23 thereof is inserted into the receiving channel 96 through an open distal end thereof that intersects the arcuate terminal edge 54 at the distal end 12B of the tool body 12. The stem 23 of the mirror head 16 is slid along the receiving channel 96 into an open distal end of the receiving cavity that faces the distal end 12 of the tool body. During such insertion of the stem 23 into the receiving cavity 99, the two grippers 98 are forced apart from one another by the outer circumference of the mirror head stem 23. Insertion of the mirror head stem 23 into the receiving cavity 99 continues until the outer perimeter of the mirrored tip 24 of the mirror head 16 abuts the concavely arcuate seating surface 58 at the distal end 12B of the tool body, thus denoting the fully inserted and installed position of the mirror head 16.

The grippers are resiliently biased toward their default positions minimizing the diameter of the receiving cavity, and thus exert a frictional holding force against the external circumferential surface of the mirror head stem 23, thereby frictionally securing the installed mirror head in place on the tool body to prevent axial separation of the mirrored tip 24 from the arcuate seating surface 58 at the mirror holding portion 40 of the tool body 12. As shown in the drawings, to help guide the mirror head stem 23 along the receiving channel 96 and into the receiving cavity 99, a guide arch 102 may span arcuately over the receiving channel 96 at a location between the receiving cavity 99 and the distal end of the receiving channel, thus cooperating with the receiving channel 96 to form a guide tunnel 104 through which the stem 23 of the mirror head is guided along the receiving channel 96 into the receiving cavity 99.

The valve portion 32 of the tool body 12 is externally barrel shaped, and internally defines a cylindrical valve-bore 62 centered on a rotation axis 64 that lies orthogonal to the longitudinal axis 18 in the thickness direction T of the tool body 12. A cylindrical valve body 66 is received in the valve-bore 62 and is rotatable therein about the rotation axis 64 to define an operable control valve for controlling air flow through the tool body'. A cross-bore 68 extends through the valve body 66 on a bore axis that is coplanar with the central longitudinal axis 18 of the tool body 12. The valve body 66 is rotatable into different positions of varying alignment between the cross-bore 68 of the valve body 66 and the internal passage 20 of the tool body 12. FIG. 10 shows a fully open position of the control valve, where the cross-bore 68 is in full alignment with the internal passage 20 of the tool body so that the bore axis of the valve body 66 is coincident with the longitudinal axis 18 of the tool body 12. In a fully closed position of the control valve, the cross-bore 68 instead lies transversely of the internal passage 20 with the ends of the cross-bore 68 closed off by the opposing lateral sides of the barrel shaped valve portion 32 of the tool body 12, whereby the valve body blocks all airflow through the internal passage 20 in this closed position. In intermediate positions of the control valve, the cross-bore 68 has its two ends partially covered by the opposing lateral sides of the barrel shaped valve portion 32 of the tool body 12, yet partially open to the internal passage 20 with the bore axis obliquely skewed from the longitudinal axis 18. The control valve is thus operable to vary operation the tool between fully on, full off and intermediate states of suction.

For moving the valve body 66 between its different positions, a valve manipulator 70 is coupled to valve body 66 at the top side of the tool body and resides externally thereof for manual one-handed operation using the same hand in which the grip portion 30 of the tool body is held. The manipulator 70 features a lever 72 projecting radially outward from the valve body in a radial direction relative to the valve's rotation axis 64, and an end bar 74 that hangs from a free end of the lever 72 in an orientation parallel to the valve's rotation axis 64 at a location alongside the tool body 12 at one of its lateral sides 46A. The end bar 74 serves as both a stop for limiting the angular rotation of the valve body 66 in either direction about the rotation axis 64 by eventual contact of the end bar 74 the respective lateral side 46A of the tool body under such rotation, and as an actuation input that the user's fingertip can push and pull longitudinally away from and back toward the grip portion 30 of the tool body in order to actuate movement of the control valve between its open and closed positions. In the illustrated embodiment, when the valve is in the closed position, the push bar 74 resides in a small notch that exists in the lateral side 46A of the tool body where grip and valve portions are connected together by a reduced-diameter neck 76 whose lesser width than the grip and valve portions of the valve body creates this notch. In the illustrated embodiment, the manipulator lever 72 has a tapered teardrop shape, a wider end of which overlies the valve body 66 and has equal diameter thereto to form an integral cap thereof. The teardrop shape narrows outwardly from the valve body 66 toward the free end of the lever 72. A user can push and pull the manipulator 70 via the end bar 74 and/or the tapered portion of the lever 72 that radiates outward from the valve body 32.

The grip portion 30 of the tool body 12 neighbouring the valve portion 32 thereof is of generally greater exterior width and thickness (i.e. larger circumference) than the joining portion 36 on the distal side of the valve portion 32. At a distal region of the grip portion 30 immediately adjacent the valve portion 32, the grip portion of the tool body is externally scalloped, for example having four concave scallops 78 respectively positioned at the tool body's four sides. Each scallop 78 has a concave curvature in both the longitudinal direction of the tool body 12, and in the circumferential thereof around the longitudinal axis 18. The tool body 12 can be comfortably gripped in the hand of a user, for example in a pen-grip fashion pinched from the lateral sides 46A, 46B of the tool body 12 between the thumb and middle finger, with the index finger placed on the top side 42, and all three of these digits of the user's hand rested conformably in a respective one of the scallops 78. The control valve can be operated by the same hand, for example though pushing and pulling of the lever 72 of the valve manipulator 70 by the index finger, or pushing and pulling of the end bar 74 of the valve manipulator by the middle finger, or a combination of such actions.

In the illustrated embodiment, in addition to the scallops 78, the exterior of the grip portion 30 is further characterized by a set of recessed longitudinal grip channels 80, each spanning longitudinally from a respective one of the scallops 78 toward the proximal end of the tool body. In the illustrated example, these grip channels 80 each extend a full length of the grip portion 30 to the coupling portion 28, where the hose connector 14 is mounted. In the illustrated example, the recessed grip channels 80 are concavely contoured in the circumferential direction, and are tapered in the longitudinal direction, in a manner growing wider toward the proximal end 12A of the tool body.

The coupling portion 28 features an external angular stop shoulder 82 at the distal end of the coupling portion 28 from which the neighbouring grip portion 30 longitudinally extends. The stop shoulder 82 juts radially outward from the neighbouring proximal end of the grip portion 30. From this stop shoulder 82, a remainder of the coupling portion 28 takes the form of an externally threaded hollow cylinder 84 of lesser outer diameter than the stop shoulder 82. An internal bore of this threaded hollow cylinder 84 defines a respective section of the internal passage 20, while the external threading of the cylinder 84 is capable of receiving threaded attachment of an internally threaded retention cap 86 of a two-piece hose connector 14. The other piece of the hose connector 14 is a connector body 88 having a hose receiver 90 in the form a hollow tubular shaft, and a ball 92 of spherically convex exterior contour attached to a proximal end of the hose receiver 90. An axial bore 91 passes fully through the connector body on a longitudinal axis of the hose receiver's tubular shape.

An annular rim 84A of the hollow cylinder 84 of the tool body's coupling portion 28 at the proximal end 12A of the tool body has a concavely spherical contour of generally equal radius of curvature to the spherically convex exterior of the ball 92 of the connector body 88. A proximal end of the retention cap 86 has an in-turned flange 94 whose inner end nearest to the longitudinal axis of the tool body 12 also has a concavely spherical contour of generally equal radius of curvature to the spherically convex exterior contour of the ball 92 of the connector body 88. The spherically concave contours of the annular rim 84A of the tool body's proximal end 12A and the in-turned flange of the retention cap 86 share a common radial center when the retention cap 86 is fully threaded onto the externally threaded distal end of the tool body 12. Accordingly, when the internally threaded retention cap 86 is threaded onto the externally threaded proximal end 12A of the tool body 12, the ball 92 is captured between the spherically contoured annular rim 84A of the tool body's proximal end 12A and the spherically contoured flange 94 of the retention cap 86, but is allowed to swivel about three orthogonal axes within this captured space between the retention cap 86 and proximal end 12A of the tool body. When a working end of a suction hose, whose opposing end is connected to a vacuum pump, is fitted over the hose receiver 90 in order to apply suction to the internal passage 20 of the tool body via the bore 91 of the connector body 88, the ball 92 allows swivelling of the hose receiver 90 and attached working end of the suction hose relative to the tool body 12, thus helping to prevent kinking of the hose during use of the tool at varying angles.

While this swivelling hose connector is beneficial for such purposes, other embodiments may forgo the swivelling hose connector 14 of the illustrated embodiment, and instead employ a rigidly static hose connector, for example as a seamlessly integral part of the tool body 12 at the location thereof occupied in the illustrated embodiment by the externally threaded hollow cylinder 84 that receives the retention cap 86 of the two-piece swivelling hose connector. In such alternative embodiments, the suction hose's working end would instead bit fitted directly to the proximal end of the tool body 12 itself. Such alternate embodiments employing a static hose connector may benefit from reduced cost relatively to the added componentry count and mechanical complexity of the illustrated embodiment. It will also be appreciated that while the swivelling hose connector is illustrated specifically in the context of a combined mirror holder and suction device, the novel hose connector may also be used on any suction device, whether a mirror-supporting suction device or not.

Since various modifications can be made in my invention as herein above described, and many apparently widely different embodiments of same made, it is intended that all matter contained in the accompanying specification shall be interpreted as illustrative only and not in a limiting sense.

1. A combination mirror holder and suction device for dental applications, said device comprising:
an elongated tool body having a proximal end, a distal end residing opposite thereto in a longitudinal direction of said tool body denoted by a longitudinal axis thereof, said tool body having opposing top and bottom sides residing oppositely of one another in a thickness direction orthogonally transverse to said longitudinal axis, and opposing lateral sides residing opposite one another in a width direction orthogonally transverse to both said longitudinal axis and said first direction;
a hollow internal passage running longitudinally and internally of said tool body on the longitudinal axis thereof;
one or more suction ports fluidly communicating said hollow internal passage with an exterior of the tool body at or adjacent the distal end thereof, of which at least one of said one or more suction ports resides at the top side of the tool body; and
a hose connector residing at or adjacent the proximal end of the elongated tool body and fluidly communicating with said hollow internal passage to enable connection of a suction hose from a vacuum pump to apply suction at said suction port;
wherein for the purpose of removably supporting a replaceable dental mirror head that is composed of stem and a mirrored tip attached to one end of said stem, said distal end of the tool body comprises a concavely arcuate seating edge in which the tool body terminates at the distal end thereof, and whose concavity faces distally of the tool body in a reference plane of obliquely oriented relation to the longitudinal axis of the tool body to accept seated placement of a stem-adjacent region of an outer circumference of the mirrored tip of the mirror head against said curved seating edge in a position facing a reflective surface of said mirrored tip outwardly away from the top side of the tool body.

2. The device of claim 1 wherein the tool body comprises, at the bottom side thereof and at a proximally spaced distance from the concavely arcuate seating edge, a longitudinally oriented receiving cavity having an open end facing longitudinally toward the concavely arcuate seating edge accommodate the stem of the mirror head.

3. (canceled)

4. (canceled)

5. (canceled)

6. (canceled)

7. The device of claim 1 wherein the distal end of the tool body comprises a plurality of support nubs projecting distally from said concavely arcuate seating edge to brace a backside of the mirrored tip of the mirror head.

8. A combined mirror holder and suction device for dental applications, said device comprising:
an elongated tool body having a proximal end, a distal end residing opposite thereto in a longitudinal direction of said tool body;
a hollow internal passage running longitudinally and internally of said tool body;
at least one suction port fluidly communicating said hollow internal passage with an exterior of the tool body at or adjacent the distal end thereof;
a hose connector residing at or adjacent the proximal end of the elongated tool body and fluidly communicating with said hollow internal passage to enable connection of a suction hose from a vacuum pump to apply suction at said suction port; and
a control valve installed on said tool body at an intermediate location along the hollow passage, said control valve comprising an external manipulator situated outside the tool body and a valve body that resides within the hollow passage and is movable via the external manipulator to vary airflow through the hollow passage;
wherein:
the tool body, at or adjacent the distal end thereof, comprises a mirror holding portion that holds or is configured to hold a mirror in a working position facing outwardly from the top side of the tool body at or adjacent the distal end thereof; and
the tool is further characterized by inclusion of one or more of the following features:
(a) the tool body, at a gripping portion thereof that proximally neighbours the control valve and spans longitudinally therefrom toward the proximal end of the tool body, has an enlarged circumference relative to a joining portion of the tool body that distally neighbours the control valve and spans longitudinally therefrom toward the distal end of the tool body; and/or
(b) said hose connector is coupled to the tool body in a swiveling fashion swivelable about three orthogonal axes to enable tilting of said hose connector in multiple directions relative to the longitudinal axis of the tool body.

9. The device of claim 8 characterized by inclusion of at least feature (a), and wherein the gripping portion, at least at a distal region thereof adjacent the control valve, comprises a set of external scallops therein for finger gripping purposes, and said scallops are concavely contoured in both the longitudinal direction denoted by the longitudinal axis, and in a circumferential direction around the longitudinal axis.

10. (canceled)

11. The device of claim 8 wherein the gripping portion, at least at a distal region thereof adjacent the control valve, comprises a set of external scallops therein for finger gripping purposes, and the gripping portion also comprises a set of recessed longitudinal grip channels each spanning longitudinally from a respective one of the scallops at the distal region of the gripping portion toward the proximal end of the tool body.

12. The device of claim 8 wherein the manipulator of the control valve comprises a stop feature thereon that is positioned to abut against a stop shoulder at a distal end of the gripping region in a default position of the control valve.

13. (canceled)

14. The device of claim 8 characterized by inclusion of at least feature (b), and wherein said hose connector has an externally exposed hose receiver for connection of the suction hose, and an internally housed ball attached to an end said hose receiver and captured in swivelling fashion between the tool body and a retention cap attached to the proximal end of the tool body.

15. The device of claim 14 wherein the proximal end of the tool body is externally threaded and comprises a spherically concave rim spanning circumferentially around the hollow internal passage.

16. A combined mirror holder and suction device for dental applications, said device comprising:
an elongated tool body having a proximal end, a distal end residing opposite thereto in a longitudinal direction of said tool body denoted by a longitudinal axis thereof, said tool body having opposing top and bottom sides residing oppositely of one another in a thickness direction that is orthogonally transverse to said longitudinal axis, and opposing lateral sides residing opposite one another in a width direction that is orthogonally transverse to both said longitudinal axis and said first direction;
a hollow internal passage running longitudinally and internally of said tool body on the longitudinal axis thereof;
a plurality of suction ports fluidly communicating said hollow internal passage with an exterior of the tool body near the distal end thereof; and
a hose connector residing at or adjacent the proximal end of the elongated tool body and fluidly communicating with said hollow internal passage to enable connection of a suction hose from a vacuum pump to apply suction at said plurality of suction ports;
wherein:
the tool body, at or adjacent the distal end thereof, comprises a mirror holding portion that holds or is configured to hold a mirror in a working position situated distally of the suction ports and facing outwardly from the top side of the tool body at or adjacent the distal end thereof; and
the tool is further characterized by inclusion of one or more of the following features:
(a) said plurality of suction ports comprises a pair of primary suction ports penetrating a top wall of the tool body on the top side thereof at a location situated adjacent to the mirror holding portion at a proximal side thereof nearest to the proximal end of the tool body; and
(b) at a region of said tool body approaching and connected to said mirror-holding portion, one or more of: (i) a concave external curvature at the top side of the tool body; (ii) a tapered shape by which a depth of the internal passage tapers toward the distal end of the tool body; and/or (iii) a flared shape by which a combined wall height of the bottom and lateral sides of the tool body measured in the thickness direction increases toward the distal end of the tool body.

17. The device of claim 16 characterized by inclusion of feature (a), and wherein the pair of primary suction ports are delimited from one another by an intact strip of the top wall that forms a central divider rib between the pair of suction ports.

18. The device of claim 17 wherein said divider rib, over a majority length thereof, is unconnected to a bottom wall of the tool body that resides at the bottom side thereof and lies opposite the top wall thereof.

19. The device of claim 17 wherein said divider rib, at a distal end thereof opposite the proximal end of the tool body, is connected to the mirror holding portion of the tool body.

20. The device of claim 19 wherein said divider rib, at least at a distal portion thereof that approaches and connects to the mirror holding portion, is obliquely sloped relative to the longitudinal axis of the tool body

21. (canceled)

22. The device of claim 16 characterized by inclusion of features (a), and wherein said primary suction ports reside in a flared portion of the tool body at which measurement of the tool body increases toward the distal end of the tool body in at least one of either said combined wall height measured in the thickness direction, and/or a width of the tool body measured in said width direction.

23. The device of claim 22 wherein said flared distal portion of the tool body is concavely contoured at the top side thereof.

24. (canceled)

25. The device of claim 22 wherein, at the flared portion, the internal passage has a varying depth that tapers in the thickness direction moving longitudinally toward the distal end of the tool body.

26. The device of claim 16 characterized by inclusion of feature (a), and wherein, at or near a region of the tool body occupied by the primary suction ports, an exterior of the tool body is concavely contoured at the top side thereof.

27. The device of claim 16 characterized by inclusion of feature (a), and wherein at or near a region of the tool body occupied by the primary suction ports, the internal passage has a varying depth that tapers in the thickness direction moving longitudinally toward the distal end of the tool body.

28. The device of claim 16 characterized by inclusion of feature (a), and wherein said primary suction ports each grow wider toward the distal end of the tool body.

29. The device of claim 16 characterized by inclusion of feature (a), and wherein said plurality of suction ports further comprise secondary suction ports that are of lesser size than said primary suction ports, and that respectively communicate with the primary suction ports and with the exterior of the tool body at the bottom side thereof.

30. The device of claim 29 wherein said secondary suction ports communicate with the primary suction ports at or adjacent distal ends thereof nearest to the mirror holding portion of the tool body.

31. The device of claim 29 wherein said secondary suction ports are narrow slot-shaped openings.

32. The device of claim 29 wherein said secondary suction ports curve along a concave terminal edge of the mirror holding portion of the tool body.

33. (canceled)

34. The device of claim 16 characterized by inclusion of feature (b)(i).

35. The device of claim 16 characterized by inclusion of feature (b)(ii).

36. The device of claim 16 characterized by inclusion of feature (b)(iii).

37. The device of claim 16 wherein said region of said tool body approaching and connected to said mirror-holding portion is further characterized by a flared width of the tool body that is measured in said width direction and increases toward said distal end of the tool body.
